(19) United States
(12) Patent Application Publication
(10) Pub. No.: US 2013/0022565 A1
(43) Pub. Date: Jan. 24, 2013

(54) BLEACHING COMPOSITION COMPRISING A PEROXYGENATED SALT IN A BASE VERY RICH IN FATTY SUBSTANCES

(71) Applicants: Damarys Braida-ValeRio, Paris (FR); Amer Alkahwaji, Paris (FR); Estelle Guerrini, Saint Cloud (FR)

(72) Inventors: Damarys Braida-ValeRio, Paris (FR); Amer Alkahwaji, Paris (FR); Estelle Guerrini, Saint Cloud (FR)

(73) Assignee: L'OREAL, Paris (FR)

(21) Appl. No.: 13/638,943

(22) PCT Filed: Mar. 30, 2011
(86) PCT No.: PCT/EP2011/054888

§ 371 Date: Oct. 2, 2012

Related U.S. Application Data
(60) Provisional application No. 61/324,048, filed on Apr. 14, 2010.

(30) Foreign Application Priority Data
Apr. 2, 2010 (FR) 1052518

(51) Int. Cl. A61K 8/92; A61Q 5/08; A45D 34/00

(52) U.S. Cl. 424/62; 132/221

(57) ABSTRACT

The present invention relates to a composition for lightening keratin fibres, comprising, in a cosmetically acceptable medium:—at least 30% by weight, relative to the total weight of the composition, of one or more fatty substances free of carboxylic acid groups;—one or more peroxygenated salts;—one or more basifying agents; and—hydrogen peroxide; the peroxygenated salts/hydrogen peroxide weight ratio being greater than or equal to 0.2. This composition makes it possible to achieve equivalent, or even higher, lightening levels with markedly lower concentrations of peroxygenated salts and/or of hydrogen peroxide and/or milder pH conditions and/or shorter leave-on times. The composition then makes it possible to obtain advantages in terms of skin comfort and fibre protection. Under equivalent application conditions, this composition makes it possible to significantly improve the bleaching performance, in particular in terms of lightening power and efficacy, when compared with standard bleaching compositions.

The present invention relates to a composition for lightening keratin fibres, and in particular human keratin fibres such as the hair, comprising at least 30% by weight of fatty substances, at least one peroxygenated salt, at least one basifying agent and hydrogen peroxide, the peroxygenated salts/hydrogen peroxide weight ratio being greater than or equal to 0.2.

Processes for lightening human keratin fibres such as the hair consist in using an aqueous composition comprising at least one oxidizing agent, under alkaline pH conditions in the vast majority of cases. The role of this oxidizing agent is to degrade the melanin of the hair, which, depending on the nature of the oxidizing agent present, leads to more or less pronounced lightening of the fibres. Thus, for relatively weak lightening, the oxidizing agent is generally hydrogen peroxide. When more substantial lightening is desired, peroxygenated salts, for instance persulfates, are usually used, in the presence of hydrogen peroxide.

The current hair bleaching compositions that can achieve lightening levels going beyond 4 tones are usually formulated based on sulfates, and generally contain large amounts thereof, from about 40% to 50% by weight. They are essentially sold in the form of a powder or a paste. At the time of use, these compositions are mixed with an oxidizing composition comprising hydrogen peroxide, of which the strength and mix ratio used vary as a function of the desired final result. The mixture thus obtained generally has a high pH, at a value of about 10, and the related leave-on time for achieving satisfactory lightening levels is generally long, usually about 50 minutes. Furthermore, the strength of the oxidizing composition is usually relatively high with a concentration ranging from 30 to 40 volumes before mixing, i.e. 9% to 12% by weight.

There is a need for satisfactory efficacy for lightening products, especially in terms of lightening power, while at the same time reducing the harmful effects associated with the simultaneous presence of basifying agents and oxidizing agents such as persalts and hydrogen peroxide. These harmful effects mainly concern the degradation of the keratin fibres and the odours of the basifying agents used, such as aqueous ammonia and amines.

It is thus sought to increase the effects of the basifying agents and/or oxidizing agents in order to limit their concentrations, while at the same time having maximum lightening efficacy.

The aim of the present invention is to obtain compositions for lightening keratin fibres that are more satisfactory as regards these points.

This aim and others are achieved by the present invention, one subject of which is thus a composition for lightening keratin fibres, comprising, in a cosmetically acceptable medium:

at least 30% by weight, relative to the total weight of the composition, of one or more fatty substances free of carboxylic acid groups;

one or more peroxygenated salts;

one or more basifying agents; and

hydrogen peroxide;

the peroxygenated salts/hydrogen peroxide weight ratio being greater than or equal to 0.2.

The composition in accordance with the invention makes it possible to achieve equivalent, or even higher, lightening levels with markedly lower concentrations of peroxygenated salts and/or of hydrogen peroxide and/or milder pH conditions and/or shorter leave-on times, when compared with standard bleaching compositions. The composition in accordance with the invention then makes it possible to obtain advantages in terms of skin comfort and fibre protection.

Under equivalent application conditions, the composition according to the invention makes it possible to significantly improve the bleaching performance, in particular in terms of lightening power and efficacy, when compared with standard bleaching compositions.

The present invention also relates to a process for lightening keratin fibres using the abovementioned composition.

A subject of the invention is also a multi-compartment device containing, in each of these compartments, compositions intended to be mixed together to give the composition according to the invention, just before application to the human keratin fibres.

Other characteristics and advantages of the invention will emerge more clearly on reading the description and the examples that follow.

In the text hereinbelow, unless otherwise indicated, the limits of a range of values are included in that range.

According to one particular embodiment of the invention, the composition in accordance with the invention does not comprise any direct dye or any oxidation dye precursor (bases and couplers) usually used for the dyeing of human keratin fibres, or, if they are present, their total content does not exceed 0.005% by weight relative to the total weight of the composition. Specifically, at such a content, only the composition would be dyed, i.e. no dyeing effect would be observed on the keratin fibres.

The cosmetically acceptable medium of the composition according to the invention is a medium comprising water and/or one or more organic solvents.

Examples of organic solvents that may be mentioned include linear or branched and preferably saturated monoalcohols or diols, comprising 2 to 10 carbon atoms, such as ethanol, isopropanol, hexylene glycol (2-methyl-2,4-pentanediol), neopentyl glycol and 3-methyl-1,5-pentanediol, butylene glycol, dipropylene glycol and propylene glycol; aromatic alcohols such as benzyl alcohol or phenylethyl alcohol; polyols containing more than two hydroxyl functions, such as glycerol; polyol ethers, for instance ethylene glycol monomethyl, monoethyl or monobutyl ether, propylene glycol or ethers thereof, for instance propylene glycol monomethyl ether; and also diethylene glycol alkyl ethers, especially C₁-C₄ alkyl ethers, for instance diethylene glycol monoethyl ether or monobutyl ether, alone or as a mixture.

The organic solvents, when they are present, generally represent between 1% and 40% by weight relative to the total weight of the dye composition, and preferably between 5% and 30% by weight relative to the total weight of the dye composition.

The water concentration may range from 10% to 70% and better still from 20% to 55% of the total weight of the composition.

The composition according to the invention may be in various forms, such as in the form of liquids, creams or gels, or in any other form that is suitable for lightening keratin fibres, and especially human hair.

Advantageously, the composition according to the invention is in the form of a gel or a cream.

As indicated previously, the lightening composition according to the invention comprises at least 30% by weight of one or more fatty substances free of carboxylic acid groups.

For the purposes of the present invention, the term “fatty substance” means an organic compound that is insoluble in water at ordinary temperature (25° C.) and at atmospheric pressure (760 mmHg; i.e. 1.013×10⁵ Pa), i.e. it has a water solubility of less than 5%, preferably 1% and even more preferentially 0.1% by weight. They have in their structure at least one hydrocarbon-based chain comprising at least 6 carbon atoms or a sequence of at least two siloxane groups. In addition, the fatty substances are soluble in organic solvents under the same temperature and pressure conditions, for instance chloroform, ethanol or benzene.

The fatty substances of the invention are not oxyalkylenated.

Preferably, the fatty substances of the invention are chosen from hydrocarbons, fatty alcohols, fatty esters, silicones and fatty ethers, or mixtures thereof.

The fatty substances of the invention may be liquid or non-liquid at room temperature (25° C.) and at atmospheric pressure (760 mmHg; i.e. 1.013×10⁵ Pa).

The liquid fatty substances of the invention preferably have a viscosity of less than or equal to 2 Pa·s, better still less than or equal to 1 Pa·s and even better still less than or equal to 0.1 Pa·s at a temperature of 25° C. and at a shear rate of 1 s⁻¹.

the term “liquid hydrocarbon” means a hydrocarbon composed solely of carbon and hydrogen atoms, which is liquid at ordinary temperature (25° C.) and at atmospheric pressure (760 mmHg; i.e. 1.013×10⁵ Pa).

More particularly, the liquid hydrocarbons are chosen from:

linear or branched, optionally cyclic, C₆-C₁₆ lower alkanes. Examples that may be mentioned include hexane, undecane, dodecane, tridecane, and isoparaffins, for instance isohexadecane, isododecane and isodecane,

linear or branched hydrocarbons of mineral, animal or synthetic origin, containing more than 16 carbon atoms, such as volatile or non-volatile liquid paraffins, and derivatives thereof, petroleum jelly, liquid petroleum jelly, polydecenes, hydrogenated polyisobutene such as Parleam®, and squalane.

In one preferred variant, the liquid hydrocarbon(s) are chosen from volatile or non-volatile liquid paraffins, and derivatives thereof, and liquid petroleum jelly.

The term “liquid fatty alcohol” means a non-glycerolated and non-oxyalkylenated fatty alcohol that is liquid at ordinary temperature (25° C.) and at atmospheric pressure (760 mmHg; i.e. 1.013×10⁵ Pa).

Preferably, the liquid fatty alcohols of the invention comprise from 8 to 30 carbon atoms.

The liquid fatty alcohols of the invention may be saturated or unsaturated.

The saturated liquid fatty alcohols are preferably branched. They may optionally comprise in their structure at least one aromatic or non-aromatic ring. They are preferably acyclic.

More particularly, the liquid saturated fatty alcohols of the invention are chosen from octyldodecanol, isostearyl alcohol and 2-hexyldecanol.

Octyldodecanol is most particularly preferred.

These liquid unsaturated fatty alcohols have in their structure at least one double or triple bond. Preferably, the fatty alcohols of the invention bear in their structure one or more double bonds. When several double bonds are present, there are preferably 2 or 3 of them, and they may be conjugated or unconjugated.

These unsaturated fatty alcohols may be linear or branched.

They may optionally comprise in their structure at least one aromatic or non-aromatic ring. They are preferably acyclic.

More particularly, the liquid unsaturated fatty alcohols of the invention are chosen from oleyl alcohol, linolenyl alcohol, linolenyl alcohol and undecylenyl alcohol.

Oleyl alcohol is most particularly preferred.

The term “liquid fatty ester” means an ester derived from a fatty acid and/or from a fatty alcohol and that is liquid at ordinary temperature (25° C.) and at atmospheric pressure (760 mmHg; i.e. 1.013×10⁵ Pa).

The esters are preferably liquid esters of saturated or unsaturated, linear or branched C₁-C₂₆ aliphatic mono- or polyacids and of saturated or unsaturated, linear or branched C₁C₂₆ aliphatic mono- or polyalcohols, the total carbon number of the esters being greater than or equal to 10.

Preferably, for the esters of monoalcohols, at least one from among the alcohol and the acid from which the esters of the invention are derived is branched.

Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isodecyl neopentanoate and isostearyl neopentanoate.

Esters of C₄^{-C}₂₂ dicarboxylic or tricarboxylic acids and of C₁-C₂₂ alcohols and esters of mono-, di- or tricarboxylic acids and of non-sugar C₄^{-C}₂₆ di-, tri-, tetra- or pentahydroxy alcohols may also be used.

Mention may be made especially of: diethyl sebacate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

The composition may also comprise, as liquid fatty ester, sugar esters and diesters of C₆^{-C}₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from mono-, di-, tri-, tetraesters and polyesters, and mixtures thereof.

These esters may be chosen, for example, from oleates, laurates, palmitates, myristates, behenates, cocoates, stearates, linoleates, linolenates, caprates and arachidonates, or mixtures thereof such as, especially, oleopalmitate, oleostearate and palmitostearate mixed esters.

It is more particularly preferred to use monoesters and diesters and especially sucrose, glucose or methylglucose mono- or dioleates, stearates, behenates, oleopalmitates, linoleates, linolenates and oleostearates.

An example that may be mentioned is the product sold under the name Glucate® DO by the company Amerchol, which is a methylglucose dioleate.

Finally, natural or synthetic esters of mono-, di- or triacids with glycerol may also be used.

Among these, mention may be made of plant oils.

As oils of plant origin or synthetic triglycerides that may be used in the composition of the invention as liquid fatty esters, examples that may be mentioned include:

triglyceride oils of plant or synthetic origin, such as liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, sunflower oil, castor oil, avocado oil, caprylic/capric acid triglycerides, for instance those sold by the company Stéarineries Dubois or those sold under the names Miglyol® 810, 812 and 818 by the company Dynamit Nobel, jojoba oil and shea butter oil;

Liquid fatty esters derived from monoalcohols will preferably be used as esters according to the invention.

Isopropyl myristate and isopropyl palmitate are particularly preferred.

The term “liquid silicone” means an organopolysiloxane that is liquid at ordinary temperature (25° C.) and at atmospheric pressure (760 mmHg; i.e. 1.013×10⁵ Pa).

Preferably, the silicone is chosen from liquid polydialkylsiloxanes, especially liquid polydimethylsiloxanes (PDMS) and liquid polyorganosiloxanes comprising at least one aryl group.

These silicones may also be organomodified. The organomodified silicones that can be used in accordance with the invention are silicones as defined above and comprising in their structure one or more organofunctional groups attached via a hydrocarbon-based group.

Organopolysiloxanes are defined in greater detail in Walter Noll's “Chemistry and Technology of Silicones” (1968) Academic Press. They may be volatile or non-volatile.

When they are volatile, the silicones are more particularly chosen from those having a boiling point of between 60° C. and 260° C., and even more particularly from:

(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Silicone Volatile® 7158 by Union Carbide, Silbione® 70045 V5 by Rhodia, and dodecamethylcyclopentasiloxane sold under the name Silsoft 1217 by Momentive Performance Materials, and mixtures thereof.

Mention may also be made of cyclocopolymers of the dimethylsiloxane/methylalkylsiloxane type, such as Silicone Volatile® FZ 3109 sold by the company Union Carbide, of formula:

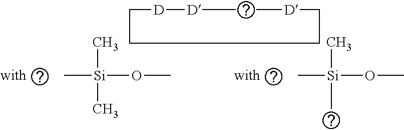

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”. The viscosity of the silicones is measured at 25° C. according to ASTM standard 445 Appendix C.

Non-volatile polydialkylsiloxanes may also be used.

These silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:

the Silbione® oils of the 47 and 70 047 series or the Mirasil® oils sold by Rhodia, for instance the oil 70 047 V 500 000;

the oils of the Mirasil® series sold by the company Rhodia;

the oils of the 200 series from the company Dow Corning, such as DC200 with a viscosity of 60 000 mm²/s;

the Viscasil® oils from General Electric and certain oils of the SF series (SF 96, SF 18) from General Electric.

Mention may also be made of polydimethylsiloxanes containing dimethylsilanol end groups known under the name Dimethiconol (CTFA), such as the oils of the 48 series from the company Rhodia.

Among the silicones containing aryl groups are polydiarylsiloxanes, especially polydiphenylsiloxanes and polyalkylarylsiloxanes. Examples that may be mentioned include the products sold under the following names:

the Silbione® oils of the 70 641 series from Rhodia;

the oils of the Rhodorsil® 70 633 and 763 series from Rhodia;

the oil Dow Corning 556 Cosmetic Grade Fluid from Dow Corning;

the silicones of the PK series from Bayer, such as the product PK20;

certain oils of the SF series from General Electric, such as SF 1023, SF 1154, SF 1250 and SF 1265.

The organomodified liquid silicones may especially contain polyethyleneoxy and/or polypropyleneoxy groups. Mention may thus be made of the silicone KF-6017 proposed by Shin-Etsu, and the oils Silwet® L722, and L77 from the company Union Carbide.

The liquid fatty acids are preferably unsaturated and/or branched fatty acids. Mention may be made in particular of oleic acid.

The liquid fatty esters are chosen from liquid dialkyl ethers such as dicaprylyl ether.

The fatty substances may be non-liquid at room temperature and at atmospheric pressure.

The term “non-liquid” preferably means a solid compound or a compound that has a viscosity of greater than 2 Pa·s at a temperature of 25° C. and at a shear rate of 1 s⁻¹.

More particularly, the non-liquid fatty substances are chosen from fatty alcohols, esters of fatty acids and/or of fatty alcohols, non-silicone waxes, silicones and fatty ethers, which are non-liquid and preferably solid.

The non-liquid fatty alcohols that are suitable for use in the invention are more particularly chosen from saturated or unsaturated, linear or branched alcohols comprising from 8 to 30 carbon atoms. Mention may be made, for example, of cetyl alcohol, stearyl alcohol and a mixture thereof (cetylstearyl alcohol).

As regards the non-liquid esters of fatty acids and/or of fatty alcohols, mention may be made especially of solid esters derived from C₉-C₂₆ fatty acids and from C₉-C₂₆ fatty alcohols.

Among these esters, mention may be made of octyldodecyl behenate; isocetyl behenate; cetyl lactate; stearyl octanoate; octyl octanoate; cetyl octanoate; decyl oleate; myristyl stearate; octyl palmitate; octyl pelargonate; octyl stearate; alkyl myristates such as cetyl, myristyl or stearyl myristate; hexyl stearate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may be made especially of: diethyl sebacate; diisopropyl sebacate; diisopropyl adipate; di-n-propyl adipate; dioctyl adipate; dioctyl maleate.

Among all the additional esters mentioned above, it is preferred to use myristyl, cetyl or stearyl palmitates, alkyl myristates such as cetyl myristate, and stearyl or myristyl myristate.

The (non-silicone) wax(es) are chosen especially from carnauba wax, candelilla wax, esparto grass wax, paraffin wax, ozokerite, plant waxes, for instance olive wax, rice wax, hydrogenated jojoba wax or the absolute waxes of flowers such as the essential wax of blackcurrant sold by the company Bertin (France), animal waxes, for instance beeswaxes, or modified beeswaxes (cerabellina); other waxes or waxy starting materials that may be used according to the invention are especially marine waxes such as the product sold by the company Sophim under the reference M82, and waxes of polyethylene or of polyolefins in general.

The non-liquid silicones that may be used in accordance with the invention may be in the form of waxes, resins or gums.

Preferably, the non-liquid silicone is chosen from polydialkylsiloxanes, especially polydimethylsiloxanes (PDMS), and organomodified polysiloxanes comprising at least one functional group chosen from poly(oxyalkylene) groups, amino groups and alkoxy groups.

The silicone gums that can be used in accordance with the invention are especially polydialkylsiloxanes and preferably polydimethylsiloxanes with high number-average molecular masses of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Products that can be used more particularly in accordance with the invention are mixtures such as:

mixtures formed from a polydimethylsiloxane hydroxylated at the chain end, or dimethiconol (CTFA) and from a cyclic polydimethylsiloxane also known as cyclomethicone (CTFA), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs with different viscosities, and more particularly of a PDMS gum and a PDMS oil, such as the product SF 1236 from the company General Electric. The product SF 1236 is a mixture of a gum SE 30 defined above, with a viscosity of 20 m²/s and of an oil SF 96 with a viscosity of 5×10⁻⁶ m²/s. This product preferably comprises 15% of gum SE 30 and 85% of an oil SF 96.

The organopolysiloxane resins that can be used in accordance with the invention are crosslinked siloxane systems containing the following units:

R2SiO2/2, R3SiO1/2, RSiO3/2 and SiO4/2

in which R represents an alkyl containing 1 to 16 carbon atoms. Among these products, the ones that are particularly preferred are those in which R denotes a C₁-C₄ lower alkyl group, more particularly methyl.

Among these resins, mention may be made of the product sold under the name Dow Corning 593 or those sold under the names Silicone Fluid SS 4230 and SS 4267 by the company General Electric, which are silicones of dimethyl/trimethylsiloxane structure.

Mention may also be made of the trimethyl siloxysilicate type resins sold in particular under the names X22-4914, X21-5034 and X21-5037 by the company Shin-Etsu.

Among the additional organomodified silicones, mention may be made of polyorganosiloxanes comprising:

substituted or unsubstituted amino groups, for instance the products sold under the names Q2 8220 and Dow Corning 929 or 939 by the company Dow Corning. The substituted amine groups are, in particular, C₁-C₄ aminoalkyl groups;
alkoxylated groups, for instance the product sold under the names Abil Wax® 2428, 2434 and 2440 by the company Goldschmidt.

The non-liquid fatty esters are chosen from dialkyl ethers and especially dicetyl ether and distearyl ether, alone or as a mixture.

Preferably, the compositions of the invention contain one or more fatty substances that are liquid at ordinary temperature (25° C.) and at atmospheric pressure (760 mmHg; i.e. 1.013×10⁵ Pa), optionally combined with one or more fatty substances that are non-liquid under the same conditions.

Preferably, the fatty substance is chosen from liquid petroleum jelly, polydecenes and liquid esters of fatty acids and/or of fatty alcohols, or mixtures thereof.

The composition according to the invention comprises at least 30% by weight of fatty substance.

According to one particular embodiment of the invention, the composition comprises at least 35% by weight of fatty substance. The composition according to the invention more particularly has a fatty substance content ranging from 30% to 80% by weight, better still from 30% to 60% by weight and even more preferentially from 35% to 60% by weight relative to the weight of the composition.

The composition according to the invention also comprises one or more peroxygenated salts.

The peroxygenated salts(s) present in the composition in accordance with the invention may be chosen, for example, from persulfates, perborates, percarbonates and peroxides of alkali metals or of alkaline-earth metals, and mixtures thereof. Persulfates and mixtures thereof will preferably be used, and more preferentially sodium, potassium and ammonium persulfates, and mixtures thereof.

The concentration of peroxygenated salts in the composition in accordance with the invention is generally between 1% and 35% by weight and preferably between 3% and 20% by weight relative to the total weight of the composition.

The composition according to the invention also comprises one or more basifying agents.

This agent may be chosen from mineral or organic or hybrid basifying agents, or mixtures thereof.

The mineral basifying agent(s) are preferably chosen from aqueous ammonia, alkali metal carbonates or bicarbonates such as sodium or potassium carbonates or bicarbonates, sodium or potassium hydroxide, and sodium or potassium metasilicate, and mixtures thereof.

The organic basifying agent(s) are preferably chosen from organic amines with a pKb at 25° C. of less than 12, preferably less than 10 and even more advantageously less than 6. It should be noted that it is the pKb corresponding to the function of highest basicity.

Hybrid compounds that may be mentioned include the salts of the amines mentioned previously with acids such as carbonic acid or hydrochloric acid.

The abovementioned organic amines, with a pKb at 25° C. of less than 12 are chosen, for example, from alkanolamines, oxyethylenated and/or oxypropylenated ethylenediamines, amino acids and the compounds of formula (I) below:

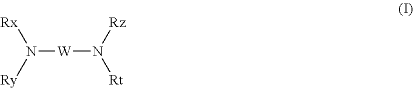

in which W is a C₁-C₆ alkylene residue optionally substituted with a hydroxyl group or a C₁-C₆ alkyl radical; Rx, Ry, Rz and Rt, which may be identical or different, represent a hydrogen atom or a C₁-C₆ alkyl, C₁-C₆ hydroxyalkyl or C₁-C₆ aminoalkyl radical.

Examples of such amines that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁-C₈ alkyl groups bearing one or more hydroxyl radicals.

Alkanolamines such as mono-, di- or tri- alkanolamines comprising from one to three identical or different C₁-C₄ hydroxyalkyl radicals are in particular suitable for performing the invention.

Among the compounds of this type, mention may be made of monoethanolamine, diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethylamino)methane.

More particularly, the amino acids that may be used are of natural or synthetic origin, in L, D or racemic form, and comprise at least one acid function chosen more particularly from carboxylic acid, sulfonic acid, phosphonic acid and phosphoric acid functions. The amino acids may be in their neutral or ionic form.

As amino acids that may be used in the present invention, mention may be made especially of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.

Advantageously, the amino acids are basic amino acids comprising an additional amine function optionally included in a ring or in a ureido function.

Such basic amino acids are preferably chosen from those corresponding to formula (II) below:

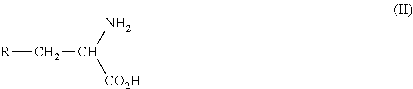

in which R denotes a group chosen from:

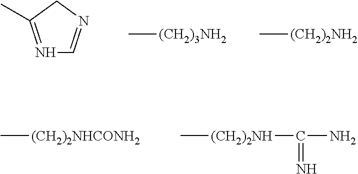

The compounds corresponding to formula (II) are histidine, lysine, arginine, ornithine and citrulline.

The organic amine may also be chosen from organic amines of heterocyclic type. Besides histidine that has already been mentioned in the amino acids, mention may be made in particular of pyridine, piperidine, imidazole, triazole, tetrazole and benzimidazole.

The organic amine may also be chosen from amino acid dipeptides. As amino acid dipeptides that may be used in the present invention, mention may be made especially of carnosine, anserine and baleine.

The organic amine may also be chosen from compounds comprising a guanidine function. As amines of this type that may be used in the present invention, besides arginine that has already been mentioned as an amino acid, mention may be made especially of creatine, creatinine, 1,1-dimethylguanidine, 1,1-diethylguanidine, glycocyamine, metformin, agmatine, N-amidinoalanine, 3-guanidinopropionic acid, 4-guanidinobutyric acid and 2-([amino(imino)methyl]amino)ethane-1-sulfonic acid.

Mention may be made in particular of the use of guanidine carbonate or monoethanolamine hydrochloride as hybrid compounds.

The organic amine is preferably chosen from alkanolamines and/or basic amino acids, more advantageously from alkanolamines.

Even more preferentially, the organic amine is monoethanolamine.

The pH of the composition according to the invention is advantageously between 7 and 12 and preferably between 8 and 11, limits inclusive.

It may be adjusted to the desired value by means of acidifying or basifying agents usually used in the lightening of keratin fibres.

The basifying agents are, for example, those described previously.

Examples of acidifying agents that may be mentioned include mineral or organic acids, for instance hydrochloric acid, orthophosphoric acid, carboxylic acids, for instance tartaric acid, citric acid or lactic acid, or sulfonic acids.

The composition according to the invention also comprises a hydrogen peroxide.

Hydrogen peroxide is present in the composition in accordance with the invention in an amount of between 1% and 12% by weight relative to the total weight of the composition.

The peroxygenated salts/hydrogen peroxide weight ratio is greater than or equal to 0.2. The said ratio preferably ranges from 0.2 to 10, better still from 0.5 to 5 and better still from 1 to 3.

The composition according to the invention may also comprise one or more surfactants.

Preferably, the surfactant(s) are selected from nonionic surfactants and anionic surfactants.

The term “anionic surfactant” means a surfactant comprising, as ionic or ionizable groups, only anionic groups. These anionic groups are preferably chosen from the following groups: CO₂H, CO₂⁻, SO₃H, SO₃⁻, OSO₃^{-H, OSO}₃⁻, H₂PO₃,HPO₃⁻, PO₃²⁻, H₂PO₂, HPO₂, HPO₂⁻, PO₂⁻, POH, PO⁻.

As examples of anionic surfactants that may be used in the composition according to the invention, mention may be made of alkyl sulfates, alkyl ether sulfates, alkylamido ether sulfates, alkylarylpolyether sulfates, monoglyceride sulfates, alkylsulfonates, alkylamidesulfonates, alkylarylsulfonates, alpha-olefin sulfonates, paraffin sulfonates, alkylsulfosuccinates, alkylether sulfosuccinates, alkylamide sulfosuccinates, alkylsulfoacetates, acylsarcosinates, acylglutamates, alkylsulfosuccinamates, acylisethionates and N-acyltaurates, salts of alkyl monoesters of polyglycoside-polycarboxylic acids, acyllactylates, D-galactoside-uronic acid salts, alkyl ether carboxylic acid salts, alkylaryl ether carboxylic acid salts, alkylamido ether carboxylic acid salts; and the corresponding non-salified forms of all these compounds; the alkyl and acyl groups of all these compounds comprising from 6 to 24 carbon atoms and the aryl group denoting a phenyl group.

These compounds may be oxyethylenated and then preferably comprise from 1 to 50 ethylene oxide units.

The salts of C₆-C₂₄ alkyl monoesters of polyglycoside-polycarboxylic acids can be selected from C₆-C₂₄ alkyl polyglycoside-citrates, C₆-C₂₄ alkyl polyglycoside-tartrates and C₆-C₂₄ alkyl polyglycoside-sulfosuccinates.

When the anionic surfactant(s) are in salt form, they may be chosen from alkali metal salts such as the sodium or potassium salt and preferably the sodium salt, the ammonium salts, the amine salts and in particular amino alcohol salts or the alkaline-earth metal salts such as the magnesium salt.

Examples of aminoalcohol salts that may especially be mentioned include mono-, di- and triethanolamine salts, mono-, di- or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts, and in particular sodium or magnesium salts, are preferably used.

The nonionic surfactants are more particularly chosen from monooxyalkylenated or polyoxyalkylenated, monoglycerolated or polyglycerolated nonionic surfactants. The oxyalkylene units are more particularly oxyethylene or oxypropylene units, or a combination thereof, preferably oxyethylene units.

Examples of oxyalkylenated nonionic surfactants that may be mentioned include:

oxyalkylenated (C₈-C₂₄)alkylphenols,

saturated or unsaturated, linear or branched, oxyalkylenated C₈-C₃₀ alcohols,

saturated or unsaturated, linear or branched, oxyalkylenated C₈-C₃₀ amides,

esters of saturated or unsaturated, linear or branched, C₈-C₃₀ acids and of polyethylene glycols,

polyoxyethylenated esters of saturated or unsaturated, linear or branched, C₈-C₃₀ acids and of sorbitol,

saturated or unsaturated, oxyethylenated plant oils,

oxyalkylenated silicones,

condensates of ethylene oxide and/or of propylene oxide, inter alia, alone or as mixtures.

The surfactants contain a number of moles of ethylene oxide and/or of propylene oxide of between 1 and 100, preferably between 2 and 50 and preferably between 2 and 30. Advantageously, the nonionic surfactants do not comprise any oxypropylene units.

In accordance with one preferred embodiment of the invention, the oxyalkylenated nonionic surfactants are chosen from oxyethylenated C₈-C₃₀ alcohols comprising from 1 to 100 mol of ethylene oxide; polyoxyethylenated esters of linear or branched, saturated or unsaturated C₈-C₃₀ acids and of sorbitol comprising from 1 to 100 mol of ethylene oxide.

As examples of monoglycerolated or polyglycerolated nonionic surfactants, monoglycerolated or polyglycerolated C₈-C₄₀ alcohols are preferably used. In particular, the monoglycerolated or polyglycerolated C₈-0₄₀ alcohols correspond to the following formula:

RO4—[CH₂—CH(CH₂OH)—O]ₘ—H

in which R represents a linear or branched C₈-C₄₀ and preferably C₈-C₃₀ alkyl or alkenyl radical, and m represents a number ranging from 1 to 30 and preferably from 1 to 10.

As examples of compounds that are suitable in the context of the invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oley Ether), oleyl alcohol containing 2 mol of glycerol (INCI name: Polyglyceryl-2 Oley Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

Among the monoglycerolated or polyglycerolated alcohols, it is more particularly preferred to use the C₈/C₁₀ alcohol containing 1 mol of glycerol, the C₁₀/C₁₂ alcohol containing 1 mol of glycerol and the C₁₂ alcohol containing 1.5 mol of glycerol.

Preferably, the surfactant optionally present in the composition is a nonionic surfactant.

The surfactant content in the composition more particularly represents from 0.1% to 50% by weight and preferably from 0.5% to 30% by weight relative to the weight of the composition.

The composition in accordance with the invention may also contain various adjuvants conventionally used in hair lightening compositions, such as anionic, cationic, nonionic, amphoteric or zwitterionic polymers or mixtures thereof; antioxidants; penetrants; sequestrants; fragrances; dispersants; film-forming agents; ceramides; preserving agents; opacifiers.

The above adjuvants are generally present in an amount for each of them of between 0.01% and 20% by weight relative to the weight of composition.

The composition may comprise one or more fumed silicas.

The fumed silicas may be obtained by high-temperature hydrolysis of a volatile silicon compound in an oxhydric flame, producing a finely divided silica. This process makes it possible especially to obtain hydrophilic silicas having a large number of silanol groups at their surface. Such hydrophilic silicas are sold, for example, under the names Aerosil 130®, Aerosil 200®, Aerosil 255®, Aerosil 300® and Aerosil 380® by the company Degussa, and Cab-O-Sil HS-5®, Cab-O-Sil EH-5®, Cab-O-Sil LM-130®, Cab-O-Sil MS-55® and Cab-O-Sil M-5® by the company Cabot.

It is possible to chemically modify the surface of the silica via chemical reaction in order to reduce the number of silanol groups. It is especially possible to substitute silanol groups with hydrophobic groups: a hydrophobic silica is then obtained.

The hydrophobic groups may be:

trimethylsiloxyl groups, which are obtained especially by treating fumed silica in the presence of hexamethyldisilazane. Silicas thus treated are known as “Silica silylate” according to the CTFA (6th edition, 1995). They are sold, for example, under the references Aerosil R812® by the company Degussa and Cab-O-Sil TS-530® by the company Cabot.

dimethylsilyloxyl or polydimethylsiloxane groups, which are especially obtained by treating fumed silica in the presence of polydimethylsiloxane or dimethyldichlorosilane. Silicas thus treated are known as “Silica dimethyl silylate” according to the CTFA (6th edition, 1995). They are sold, for example, under the references Aerosil R972® and Aerosil R974® by the company Degussa, and Cab-O-Sil TS-610® and Cab-O-Sil TS-720® by the company Cabot.

The fumed silica preferably has a particle size that may be nanometric to micrometric, for example ranging from about 5 to 200 nm.

When it is present, the fumed silica represents from 1% to 30% by weight relative to the weight of the composition.

The composition may also comprise one or more organic thickeners.

These thickeners may be chosen from fatty acid amides (coconut monoethanolamide or diethanolamide, oxyethylenated carboxylic acid monoethanolamide alkyl ether), polymeric thickeners such as cellulose-based thickeners (hydroxyethylcellulose, hydroxypropylcellulose or carboxymethylcellulose), guar gum and derivatives thereof (hydroxypropyl guar), gums of microbial origin (xanthan gum, scleroglucan gum), acrylic acid or acrylamidopropanesulfonic acid crosslinked homopolymers and associative polymers (polymers comprising hydrophilic regions and fatty-chain hydrophobic regions (alkyl or alkenyl containing at least 10 carbon atoms) that are capable, in an aqueous medium, of reversibly combining with each other or with other molecules).

According to one particular embodiment, the organic thickener is chosen from cellulose-based thickeners (hydroxyethylcellulose, hydroxypropylcellulose or carboxymethylcellulose), guar gum and derivatives thereof (hydroxypropyl guar), gums of microbial origin (xanthan gum, scleroglucan gum) and acrylic acid or acrylamidopropanesulfonic acid crosslinked homopolymers, and preferably from cellulose-based thickeners in particular with hydroxyethylcellulose.

The content of organic thickener(s), if they are present, usually ranges from 0.01% to 20% by weight and preferably from 0.1% to 5% by weight relative to the weight of the composition.

The composition of the invention may be obtained by mixing at least two or even three different compositions, or optionally more than three different compositions. One or more of the compositions leading, by mixing, to the composition of the invention may be anhydrous. It should be noted that the composition according to the invention is prepared just before being applied to the human keratin fibres: it is then referred to as a “ready-to-use composition”.

According to a first variant, the composition according to the invention is obtained by mixing at least two compositions, a first composition comprising one or more peroxygenated salts and one or more basifying agents, and a second composition comprising hydroperoxide, the fatty substance(s) being present in the first composition and/or in the second composition and/or in a third composition.

According to a second variant, the composition according to the invention is obtained by mixing at least three compositions, a first composition comprising one or more peroxygenated salts, a second composition comprising one or more basifying agents, and a third composition comprising hydroperoxide, the fatty substance(s) being present in the first composition and/or in the second composition and/or in the third composition and/or in a fourth composition.

The ingredients of the abovementioned compositions and the contents thereof are determined as a function of the characteristics detailed previously for the final composition according to the invention.

In each of the abovementioned variants, the composition comprising the peroxygenated salts(s) is anhydrous, and may be in the form of a powder or a paste.

For the purposes of the invention, the term “anhydrous composition” means a composition having a water content of less than 5% by weight, preferably less than 2% by weight and even more preferably less than 1% by weight relative to the weight of the said composition. It should be noted that the water present in the composition is more particularly “bound water”, such as water of crystallization in salts, or traces of water absorbed by the starting materials used in the preparation of the compositions according to the invention.

In each of the abovementioned variants, the composition comprising hydrogen peroxide may be anhydrous. It is preferably an aqueous composition. It may then be in the form of an aqueous solution or in the form of a direct or inverse emulsion when it comprises one or more fatty substances.

For the purposes of the present invention, an aqueous composition comprises more than 5% by weight of water, preferably more than 10% by weight of water and even more advantageously more than 20% by weight of water.

It may also comprise one or more organic solvents chosen from those listed previously; these solvents more particularly representing, when they are present, from 1% to 40% by weight and preferably from 5% to 30% by weight relative to the weight of the oxidizing composition.

The composition comprising hydrogen peroxide also preferably comprises one or more acidifying agents. The acidifying agents are, for example, those described previously.

Usually, the pH of the composition comprising hydrogen peroxide is less than 7.

The hydrogen peroxide concentration generally ranges from 0.1% to 50%, more particularly between 0.5% and 20% and even more preferentially between 1% and 15% by weight relative to the weight of the composition comprising it.

The lightening process according to the invention consists in applying the composition according to the invention to wet or dry human keratin fibres.

The composition is then left in place for a time usually ranging from one minute to one hour and preferably from 5 minutes to 30 minutes.

The temperature during the process is, conventionally, preferably between room temperature (between 15 and 25° C.) and 80° C. and preferably between room temperature and 60° C.

After the treatment, the human keratin fibres are optionally rinsed with water, optionally washed with a shampoo and then rinsed with water, before being dried or left to dry.

A subject of the invention is similarly a multi-compartment device comprising, in a first compartment, a first composition comprising one or more peroxygenated salts and one or more basifying agents, and, in a second compartment, a second composition comprising hydrogen peroxide, the fatty substance(s) being present in the first composition and/or in the second composition and/or in a third composition contained in a third compartment, the compositions of the various compartments being intended to be mixed together to give the composition according to the invention, just before application to the human keratin fibres.

A subject of the invention is, finally, a multi-compartment device comprising, in a first compartment, a first composition comprising one or more peroxygenated salts, in a second compartment, a second composition comprising one or more basifying agents, and, in a third compartment, a third composition comprising hydrogen peroxide, the fatty substance(s) being present in the first composition and/or in the second composition and/or in the third composition and/or in a fourth composition contained in a fourth compartment, the compositions of the various compartments being intended to be mixed together to give the composition according to the invention, just before application to the human keratin fibres.

The examples that follow serve to illustrate the invention without, however, being limiting in nature.

### EXAMPLES

The following compositions are prepared (the amounts are expressed in g% of product as supplied):

Composition 1 (bleaching composition)

This composition is formed from pure ammonium persulfate.

Composition 2 (bleaching composition)

| | |
|---|---|
| Potassium persulfate | 30 |
| Ethylenediaminetetraacetic acid | 0.8 |
| Technical-grade N-oleyldihydrosphingosine | 0.01 |
| Ammonium persulfate | 15 |
| Sodium disilicate hydrate | 15 |
| Anhydrous sodium metasilicate | 5.13 |
| Titanium oxide (untreated anatase) | 1 |
| Ultramarine blue | 0.04 |
| Isopropyl myristate | 21.2 |
| Liquid petroleum jelly | 0.6 |
| White beeswax | 2 |
| Xanthan: polysaccharides: glucose/mannose/glucuronic | 1 |
| acid (40/30/30) | |
| Carboxymethyl potato starch (tuber starch), sodium salt, | 2.22 |
| sparingly crosslinked | |
| Sodium lauryl sulfate | 4 |
| Magnesium stearate | 2 |

Composition 3 (alkaline composition)

| | |
|---|---|
| Diethylenetriaminepentaacetic acid, pentasodium salt | 1.35 |
| as an aqueous solution at 40% by weight | |
| Ammonia in aqueous solution at 20% by weight | 28 |
| Monoethanolamine | 20.05 |
| Sodium metabisulfite | 0.7 |
| Hydroxyethylcellulose (MW 1 300 000) | 1.5 |
| Hexylene glycol (2-methyl-2,4-pentanediol) | 3 |
| Dipropylene glycol | 3 |
| Ethyl alcohol | 8.25 |
| Propylene glycol | 6.2 |
| Vitamin C: L-Ascorbic acid as a fine powder | 0.25 |
| Water | qs 100 |

Composition 4 (alkaline composition)

| | |
|---|---|
| Diethylenetriaminepentaacetic acid, pentasodium salt | 1.35 |
| as an aqueous solution at 40% by weight | |
| Ammonia in aqueous solution at 20% by weight | 64.35 |
| Sodium metabisulfite | 0.7 |
| Hydroxyethylcellulose (MW 1 300 000) | 1.5 |
| Hexylene glycol (2-methyl-2,4-pentanediol) | 3 |
| Dipropylene glycol | 3 |
| Ethyl alcohol | 8.25 |
| Propylene glycol | 6.2 |
| Vitamin C: L-Ascorbic acid | 0.25 |
| Water | qs 100 |

Composition 5 (alkaline composition)

| | |
|---|---|
| Diethylenetriaminepentaacetic acid, pentasodium salt | 1.35 |
| as an aqueous solution at 40% by weight | |
| Monoethanolamine | 20.05 |
| Sodium metabisulfite | 0.7 |
| Hydroxyethylcellulose (MW 1 300 000) | 1.5 |
| Hexylene glycol (2-methyl-2,4-pentanediol) | 3 |
| Dipropylene glycol | 3 |
| Ethyl alcohol | 8.25 |
| Propylene glycol | 6.2 |
| Vitamin C: L-Ascorbic acid | 0.25 |
| Water | qs 100 |

Composition 6 (anhydrous gel)

| | |
|---|---|
| Distearyldimethylammonium-modified hectorite | 3 |
| (Bentone 38 VCG) | |
| 2-Octyldodecanol | 11.5 |
| Glycol distearate | 8 |
| Liquid petroleum jelly | 64.5 |
| Propylene carbonate | 1 |
| Oxyethylenated lauryl alcohol (2 OE) | 1 |
| Oxyethylenated sorbitan monolaurate (4 OE) | 11 |

Composition 7 (oxidizing composition)

| | |
|---|---|
| Diethylenetriaminepentaacetic acid, pentasodium salt | 0.15 |
| as an aqueous solution at 40% by weight | |
| Hydrogen peroxide as an aqueous solution at 50% by weight | 18 |
| Sodium stannate | 0.04 |
| Tetrasodium pyrophosphate decahydrate | 0.03 |
| Liquid petroleum jelly | 25 |
| Poly[(dimethyliminio)-1,3-propanediyl(dimethyliminio)-1,6- | 0.25 |
| hexanediyl dichloride] as an aqueous solution at 60% by | |
| weight | |
| Non-stabilized polydimethyldiallylammonium chloride in | 0.5 |
| water at 40% by weight | |
| Glycerol | 0.5 |
| Cetylstearyl alcohol (30/70 C₁₆/C₁₈) | 8 |
| Oxyethylenated cetylstearyl alcohol (33 OE) | 3 |
| Oxyethylenated rapeseed acid amide (4 OE) | 1.3 |
| Vitamin E: DL-alpha-Tocopherol | 0.1 |
| Phosphoric acid | qs pH 2.2 |
| Water | qs 100 |

Composition 8 (oxidizing composition)

| | |
|---|---|
| Hydrogen peroxide as an aqueous solution at 50% by weight | 18 |
| Etidronic acid, tetrasodium salt, as an aqueous solution | 0.2 |
| at 30% by weight | |
| Tetrasodium pyrophosphate decahydrate | 0.04 |
| Sodium salicylate | 0.035 |
| Oxyethylenated methacrylic acid/ethyl acrylate/behenyl | 2 |
| methacrylate terpolymer (25 OE) as an aqueous emulsion | |
| Cetylstearyl alcohol (30/70 C₁₆/C₁₈) | 8 |
| Oxyethylenated cetylstearyl alcohol (33 OE) | 2 |
| Phosphoric acid | qs pH 3.0 |
| Water | qs 100 |

I/Comparison Between a Bleaching System in Accordance with the Invention and a Standard Bleaching System

Mode of Application

Compositions 1, 2, 3, 4, 6, 7 and 8 detailed above are mixed at the time of the use in the following proportions (in grams):

| | Mixture 1 | Mixture 2 |
|---|---|---|
| | (invention) | (prior art) |
| Composition 1 | 3.9 | 0 |
| Composition 2 | 0 | 1 |
| Composition 4 | 4 | 0 |
| Composition 6 | 10 | 0 |
| Composition 7 | 25 | 0 |
| Composition 8 | 0 | 1.5 |

Mixture 2 corresponds to a commercial product.

The amounts of persulfates and of fatty substance in each of the mixtures are indicated in the table below.

| | Mixture 1 | Mixture 2 |
|---|---|---|
| | (invention) | (prior art) |
| Amount of persulfates (g %) | 9.1 | 12 |
| Amount of fatty substance (g %) | 38.8 | 14.3 |

The two mixtures thus obtained are then applied to locks of natural chestnut-brown hair with a tone depth of 4, at a rate of 10 g of mixture per 1 g of hair.

The application is performed at a controlled temperature of 20° C. for 30 or 50 minutes.

The hair is then rinsed, washed with a standard shampoo and dried.

Determination of the Colour

The colorimetric measurements are taken using a Konica-Minolta CM-2600d spectrocolorimeter in the L*a*b* system.

According to this system, L*represents the luminance. The chromatic coordinates are expressed by the parameters a* and b*, a* corresponding to the red/green chromatic axis and b* to the yellow/blue chromatic axis.

ΔE, which corresponds to the colour variation between a lock of natural dark hair with a tone depth equal to 4 and a lock of bleached hair, is obtained from the following formula:

ΔE=√(L*−L₀*)²+(a*−a₀*)²+(b*−b₀*)²

in which L* represents the luminance and a* and b* the chromatic coordinates of the lock of bleached hair, whereas L₀* represents the luminance and a₀* and b₀* the chromatic coordinates of the lock of natural dark hair with a tone depth equal to 4.

| | ΔE 30 minutes | ΔE 50 minutes |
|---|---|---|
| Mixture 1 (invention) | 20 | 27 |
| Mixture 2 (prior art) | 15 | 23 |

Mixture 1 makes it possible to obtain better lightening than mixture 2, even though the persulfate concentration in mixture 2 is much higher than the persulfate concentration in mixtures 1 and 2, and even though the pH of mixture 1 is also lower than the pH of mixture 2 (9.45 versus 10.05).

The fact that the mixtures in accordance with the invention have a pH value below that of the mixture according to the prior art also affords advantages in terms of skin comfort and fibre protection.

### II/Effect of the Concentration of Fatty Substances

Compositions 1, 2, 4, 6, 7 and 8 detailed above and water are mixed at the time of the use in the following proportions (in grams):

| | Mixture 2 | Mixture 1 |
|---|---|---|
| | (prior art) | (invention) |
| Composition 1 | 3.9 | 3.9 |
| Composition 4 | 4 | 4 |
| Composition 6 | 0 | 10 |
| Composition 7 | 25 | 25 |
| Water | 10 | 0 |

The amounts of fatty substance in each of the mixtures are indicated in the table below.

| | Mixture 2 | Mixture 1 |
|---|---|---|
| | (prior art) | (invention) |
| Amount of fatty substance (9%) | 19.2 | 38.8 |

The two mixtures thus obtained are then applied to locks of natural chestnut-brown hair with a tone depth of 4, at a rate of 10 g of mixture per 1 g of hair.

The application is performed at room temperature (20° C.) for 50 minutes.

The hair is then rinsed, washed with a standard shampoo and dried.

The results obtained in terms of lightening are collated in the table below

| | ΔE after 50 minutes |
|---|---|
| Mixture 2 (prior art) | 21.5 |
| Mixture 1 (invention) | 27 |

These results show that the greater the amount of fatty substances contained in the mixture according to the invention, the better the lightening power.

In all cases, better lightening power is obtained with the mixtures in accordance with the invention.

### III/Effect of the Nature of the Alkaline Agents

Compositions 1 to 8 detailed above are mixed at the time of the use in the following proportions (in grams):

| | Mixture 1 | Mixture 2 | Mixture 3 |
|---|---|---|---|
| | (invention) | (invention) | (invention) |
| Composition 1 | 3.9 | 3.9 | 3.9 |
| Composition 3 | 0 | 4 | 0 |
| Composition 4 | 4 | 0 | 0 |
| Composition 5 | 0 | 0 | 4 |
| Composition 6 | 10 | 10 | 10 |
| Composition 7 | 25 | 25 | 25 |

The nature and amount (g %) of the alkaline agents present in each of the mixtures are indicated in the table below.

| | Mixture 1 | Mixture 2 | Mixture 3 |
|---|---|---|---|
| | (invention) | (invention) | (invention) |
| Alkaline | Aqueous ammonia | Ammonia/ | Monoethanol- |
| agents | (6%, i.e. | Monoethanol- | amine |
| | 1.2% NH₃) | amine | (1.87%) |
| | | (2.6% (0.52% | |
| | | NH₃)/1.87%) | |

The three mixtures thus obtained are then applied to locks of natural chestnut-brown hair with a tone depth of 4, at a rate of 10 g of mixture per 1 g of hair.

The application is performed at room temperature (20° C.) for 50 minutes.

The hair is then rinsed, washed with a standard shampoo and dried.

The results obtained in terms of lightening are collated in the table below.

| | ΔE*ab after 50 minutes |
|---|---|
| Mixture 1 (invention) | 27 |
| Mixture 2 (invention) | 27 |
| Mixture 3 (invention) | 25 |

These results show that the mixtures in accordance with the invention make it possible to obtain very good lightening power, irrespective of the alkaline agents used.

The fact that the mixtures in accordance with the invention have a pH value below that of the mixture according to the prior art also affords advantages in terms of skin comfort and fibre protection.

1-14. (canceled)

15. A composition for lightening keratin fibres, comprising, in a cosmetically acceptable medium:
at least about 30% by weight, relative to the total weight of the composition, of at least one fatty substance free of carboxylic acid groups;
at least one peroxygenated salt;
at least one basifying agent; and
hydrogen peroxide;
wherein the weight ratio of the at least one peroxygenated salt/hydrogen peroxide is greater than or equal to about 0.2.

16. The composition according to claim 15, wherein the at least one fatty substance is chosen from hydrocarbons, fatty alcohols, fatty esters, silicones, fatty ethers, and mixtures thereof.

17. The composition according to claim 15, wherein the at least one fatty substance is chosen from fatty substances which are liquid or non-liquid at room temperature and at atmospheric pressure.

18. The composition according to claim 15, wherein the at least one fatty substance is chosen from liquid petroleum jelly, polydecenes, liquid esters of fatty acids and/or of fatty alcohols, liquid fatty alcohols, and mixtures thereof.

19. The composition according to claim 15, wherein the at least one peroxygenated salt is chosen from alkali metal or alkaline-earth metal persulfates, perborates, percarbonates, and peroxides.

20. The composition according to claim 15, wherein the at least one peroxygenated salt is chosen from persulfates and mixtures thereof.

21. The composition according to claim 15, wherein the at least one basifying agent is chosen from mineral basifying agents, organic basifying agents, and hybrid basifying agents.

22. The composition according to claim 21, wherein the at least one basifying agent is chosen from aqueous ammonia, alkali metal carbonates or bicarbonates, sodium or potassium hydroxide, organic amines with a pKb at 25° C. of less than 12, and salts thereof.

23. The composition according to claim 21, wherein the at least one basifying agent is chosen from aqueous ammonia and alkanolamines.

24. The composition according to claim 21, in which the basifying agent is monoethanolamine.

25. The composition according to claim 15, wherein the weight ratio of the at least one peroxygenated salt/hydrogen peroxide ranges from about 0.2 to about 10.

26. The composition according to claim 25, wherein the weight ratio of the at least one peroxygenated salt/hydrogen peroxide ranges from about 0.5 to about 5.

27. The composition according to claim 25, wherein the weight ratio of the at least one peroxygenated salt/hydrogen peroxide ranges from about 1 to about 3.

28. The composition according to claim 15, wherein the content of the at least one fatty substances ranges from about 30% to about 80% by weight, relative to the weight of the composition.

29. The composition according to claim 28, wherein the content of the at least one fatty substances ranges from about 30% to about 60% by weight, relative to the weight of the composition.

30. The composition according to claim 28, wherein the content of the at least one fatty substances ranges from about 35% to about 60% by weight, relative to the weight of the composition.

31. A process for lightening keratin fibers, said process comprising applying to said keratin fibers a composition comprising, in a cosmetically acceptable medium:
at least about 30% by weight, relative to the total weight of the composition, of at least one fatty substance free of carboxylic acid groups;
at least one peroxygenated salt;
at least one basifying agent; and
hydrogen peroxide;
wherein the weight ratio of the at least one peroxygenated salt/hydrogen peroxide is greater than or equal to about 0.2.

32. A multi-compartment device comprising,
in a first compartment, a first composition comprising:
at least one peroxygenated salt;
at least one basifying agent; and
in a second compartment, a second composition comprising:
hydrogen peroxide;
wherein the device further comprises at least one fatty substance in at least one of the first composition of the first compartment, the second composition of the second compartment, or a third composition in a third compartment,
wherein the compositions of the first, second, and third compartments may be mixed together and optionally applied to keratin fibers.

33. A multi-compartment device comprising,
in a first compartment, a first composition comprising:
at least one peroxygenated salt;
in a second compartment, a second composition comprising:
at least one basifying agent;
in a third compartment, a third composition comprising:
hydrogen peroxide;
wherein the device further comprises at least one fatty substance in at least one of the first composition of the first compartment, the second composition of the second compartment, the third composition of the third compartment, or a fourth composition of a fourth compartment,
wherein the compositions of the first, second, third, and fourth compartments may be mixed together and optionally applied to keratin fibers.
